(19) United States
(12) Patent Application Publication
(10) Pub. No.: US 2013/0085570 A1
(43) Pub. Date: Apr. 4, 2013

(54) FENESTRATOR DEVICE AND RELATED METHODS

(71) Applicant: Karce Daniel Rose, Healdsburg, CA (US)

(72) Inventor: Karce Daniel Rose, Healdsburg, CA (US)

(21) Appl. No.: 13/250,714

(22) Filed: Sep. 30, 2011

(51) Int. Cl. A61F 2/10; B26F 1/02

(52) U.S. Cl. 623/15.12; 234/128

(57) ABSTRACT

Devices, kits and/or methods for, or related to, creating a plurality of openings in a skin graft or skin substitute are described. The devices can have a plurality of perforating elements extending from a perforation component that can be safely held and manipulated by a user. The user can apply sufficient force to the perforating elements against the skin graft or skin substitute to enable the extended ends of the perforating elements to pass through the skin graft or skin substitute as to create openings therethrough.

### BACKGROUND

1. Field

Embodiments disclosed herein relate generally to systems, devices, compositions of matter, and methods for preparing and/or treating skin grafts and/or skin substitute materials. More specifically, certain embodiments concern systems, devices, compositions of matter, and methods that can be implemented to rapidly perforate skin grafts and/or skin substitute materials before such grafts or substitute materials are utilized, for example, to treat a patient.

2. Description of the Related Art

The application of skin grafts and/or skin substitute materials to various skin defects is commonplace in the United States. Skin defects may be caused, for example, by trauma, thermal burns, pressure injury, radiation injury, diabetes, infection, and/or vascular disorders. Suitable donor graft material can be harvested from the tissue of a patient or can be obtained commercially from processed cadaver tissue. In addition, tissue engineered skin substitute products can also be used to treat skin defects and/or to stimulate wound healing.

### SUMMARY

The systems, devices, and methods disclosed herein each have several aspects, no single one of which is solely responsible for their desirable attributes. Without limiting the scope of the claims, some prominent features will now be discussed briefly. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description of Certain Embodiments,” one will understand how the features of the devices and methods disclosed herein can provide advantages over other known devices and methods.

Some embodiments herein relate to devices for fenestrating a material such as a graft, for example. The devices may include, for example, one or more of a perforation component having, for example, one or both of a plurality of perforating elements extending from a body and at least one alignment element extending from said body; a base component having, for example, a plurality of receiving spaces, each being configured to at least partially receive at least one of the plurality of perforating elements. The at least one alignment element may be configured, for example, to align the perforation component relative to the base component such that the plurality of perforating elements are aligned with the plurality of receiving spaces, for example, in at least a transverse direction.

The plurality of receiving spaces can be sized and/or shaped so as to receive the at least one of the plurality of perforating elements without contacting the at least one of the plurality of perforating elements. The plurality of perforating elements can include, for example, one or more perforating elements of different dimensions, for example, different transverse dimensions or lengths. In some aspects, the plurality of perforating elements can include, for example, a first set of perforating elements and at least second set of perforating elements, wherein a maximum transverse dimension of each of the first set of perforating elements is greater than a maximum transverse dimension of each of the second set of perforating elements.

The at least one alignment element may include or be, for example, a dowel, a groove, a channel, a pin, a tab, a notch, or any other like structure. It should be noted that in some aspects, the at least one alignment element can be part of or be included with the base component in addition to or rather than the perforating element. The base component may include, for example, an alignment receptacle configured to receive at least a portion of the alignment element (e.g., the dowel, etc.). It should be noted that the at least one alignment receptacle can be included with the perforation component in addition to or instead of with the perforation component. The at least one alignment element may include, for example, a sleeve or other opening, wherein the sleeve or opening is sized and shaped so as to slide over at least a portion of the base.

The devices further can include, for example, a support plate. The support plate may include, for example, a plurality of holes that can be configured to receive at least one of the perforating elements therethrough. The at least one alignment element mentioned above can be configured to align the perforation component relative to the support plate such that the plurality of perforating elements are aligned with the plurality of holes in at least a transverse direction, for example. The support plate may be sized and shaped so as to be disposed, for example, at least partially between the perforation component and the base at least when the perforating elements are at least partially received within the receiving spaces, for example. The perforation component can include, for example, at least one slot, and the support plate can include at least one tab, such that the at least one tab is configured to extend through the at least one slot so as to extend beyond the perforation component. The perforation component may have or can include a maximum lateral dimension that is at least partially greater than a maximum lateral dimension of the support plate.

The plurality of perforating elements (some or each of them) can extend in the transverse direction. The at least one alignment element can extend from the body component, for example, in the transverse direction. The perforation component can include, for example, at least one flange for use in separating the perforation component from the base.

Some embodiments relate to kits. The kits can include, for example, two or more of the following: a fenestrator device as described above or elsewhere herein; one or more fenestrator device components as described herein; and a material that is to be perforated, for example, a graft of an artificial and/or natural substance. In some aspects, the material to be perforated can include, for example, a skin graft, a skin substitute material, combinations of the same, and other like materials. The kits further can include instructions for using or operating the fenestrator device or components of the device, for example. The instructions for using the fenestrator device may include indicia formed on the fenestrator device, for example.

Also, some embodiments relate to a method or methods for perforating a material, such as a graft. The methods can include, for example, providing, obtaining or utilizing a fenestrator device having a perforation component and a base. The perforation component may include, for example, a plurality of perforating elements extending from a body; the base including a plurality of receptacles configured to at least partially receive at least one of the plurality of perforating elements; providing, obtaining or utilizing a graft; disposing the graft over at least a portion of the base; and manipulating the perforation component such that at least one of the plurality of perforating elements perforates the graft and extends into or through at least one of the plurality of receptacles. The graft may include, for example, a skin or tissue graft, a skin or tissue substitute material or support material, a material that is applied for the treatment or healing of skin or tissue, combination of the same, and other like materials.

The providing, obtaining or utilizing the fenestrator device can include providing, obtaining or utilizing a support plate having a plurality of holes configured to receive at least one of the perforating elements therethrough, and wherein manipulating the perforation component may include disposing the perforation component relative to the support plate such that the support plate is disposed at least partially between the material (e.g., the graft) and the perforation component, for example. The methods further can include aligning the perforation component relative to the base such that the plurality of perforating elements are aligned relative to the plurality of receptacles in at least a transverse direction, for example. The methods further may include perforating the graft with at least one of the plurality of perforating elements.

These, as well as other components, steps, features, objects, benefits, and advantages will now become clear from a review of the following detailed description of certain embodiments, the accompanying drawings, and the claims.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings.

FIG. 1A schematically illustrates a bottom perspective view of a non-limiting example of a perforation component.

FIG. 1B schematically illustrates a bottom plan view of a non-limiting example of the perforation component of FIG. 1A.

FIG. 2A schematically illustrates a top perspective view of a non-limiting example of a support plate.

FIG. 2B schematically illustrates a top plan view of a non-limiting example the support plate of FIG. 2A.

FIG. 3A schematically illustrates a top perspective view of a non-limiting example of a base.

FIG. 3B schematically illustrates a top plan view of a non-limiting example the base of FIG. 3A.

FIG. 4A schematically illustrates an exploded view of a non-limiting example of a fenestrator device including the perforation component of FIG. 1, the support plate of FIG. 2, and the base of FIG. 3, along with an example graft.

FIG. 4B schematically illustrates a partially exploded view of a non-limiting example of the fenestrator device of FIG. 4A showing the perforation elements of the perforation component extending through the support plate, along with an example graft.

FIG. 4C schematically illustrates a non-limiting example of the fenestrator device of FIG. 4A with the perforating elements of the perforation component disposed at least partially within the receptacles of the base.

FIG. 4D schematically illustrates a cross-sectional view of a non-limiting example of the fenestrator device of FIG. 4C taken along line 4D-4D.

FIG. 4E schematically illustrates a partially exploded view of a non-limiting example of the fenestrator device of FIGS. 4A-4D after the perforation component and support plate have been disengaged from the position illustrated in FIG. 4D.

FIG. 4F schematically illustrates a perspective view of the example graft of FIG. 4B after it has been perforated by the fenestrator device of FIGS. 4A-4E.

FIG. 5A schematically illustrates a bottom perspective view of another non-limiting example of a perforation component.

FIG. 5B schematically illustrates a bottom plan view of a non-limiting example the perforation component of FIG. 5A shown with another example of a support plate.

FIG. 6A schematically illustrates an exploded view of another non-limiting example of a fenestrator device, along with an example graft.

FIG. 6B schematically illustrates a non-limiting example of the fenestrator device of FIG. 6A with the perforating elements of the perforation component disposed at least partially within the receptacles of the base.

FIG. 6C schematically illustrates a cross-sectional view of a non-limiting example of the fenestrator device of FIG. 6B taken along line 6C-6C.

FIG. 6D schematically illustrates an exploded view of a non-limiting example of the fenestrator device of FIGS. 6A-6C, along with an example graft, after the perforation component and support plate have been disengaged from the position illustrated in FIG. 6C.

FIG. 6E schematically illustrates a non-limiting example of a graft that has been perforated by the fenestrator device of FIGS. 6A-6D.

### DETAILED DESCRIPTION OF CERTAIN EMBODIMENTS

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, and designed in a wide variety of different configurations, all of which are explicitly contemplated and made part of this disclosure.

As discussed above, skin grafts and/or tissue engineered skin substitute materials, referred to herein collectively as “grafts” for clarity, can be applied, for example, to treat various skin defects and/or to stimulate wound healing. In some instances, grafts may be applied surgically by a medical professional, e.g., a surgeon. In such instances, standard surgical procedure generally advises that the medical professional create a plurality of openings, holes, or apertures in the graft before utilizing the graft to treat a patient. The resultant fenestrated graft may allow wound exudates to pass through the graft so as to prevent separation of the graft from the patient's wound bed. Thus, the process of fenestrating a graft can reduce the likelihood that the graft may lose viability after being implanted in or onto a patient. Furthermore, fenestrating a graft may allow it to expand and cover an area larger than a similarly sized graft that has not been fenestrated (e.g., a graft that does not include a plurality of openings or apertures formed therethrough).

In some examples, fenestrating a graft may include using a scalpel or scissors to cut openings, holes, or apertures in the graft. However, such processes require valuable time and can produce inconsistent results. In other examples, a mesher device or “mesher” can be used to form incisions in a graft. Such mesher devices can include a plurality of blades on rollers through which a graft may be fed, usually on a carrier plate. Meshers can also include drums, axles, cutting devices, and/or other mechanical structures to produce a set pattern of incisions in grafts. In use, a medical professional typically mounts a graft on a carrier plate and feeds it into the mesher, which may be hand operated or motorized. When the graft emerges from the mesher, it has a pattern of incisions that allow it to stretch beyond its original dimensions and to allow wound exudates to pass therethrough.

A mesher may present several disadvantages. First, a mesher may be heavy and hard to move. Second, a mesher may be formed of materials that do not cool quickly and, thus, the device may not cool sufficiently for safe usage for several hours after the device is sterilized by heat. As a result, the sterilization of a mesher may result in delays in treating patients. Third, many mesher devices have many moving parts that need careful and time-consuming cleaning prior to sterilization. Fourth, due to a mesher device's mechanical complexity, it may consume significant training time for proper usage. Fifth, mesher devices may be among the most costly equipment in an individual medical professional's office. As a result, a mesher may be out of the financial reach of small medical facilities. Sixth, a process for manufacturing a mesher requires many steps due to the mechanical complexity of such devices. Seventh, a mesher may create too many incisions in a graft that may unnecessarily increase the fragility of the graft and make it more difficult to safely handle. Lastly, the set pattern of graft incisions produced by a mesher may be aesthetically unattractive to a patient.

Other examples of devices used for fenestrating a graft include graft cutter devices. Such devices also can present several disadvantages. For example, if inadequate force is applied or the blades are not sharp enough, the graft may not be adequately incised. This disadvantage may be exacerbated by a dulling of the blades due to repetitive contact of the blades with a support block. On the other hand, if a cutting force is too great and/or the support block is too soft, the cutting blades may penetrate the graft too deeply, causing it to become attached to the blades. Further, removing an adherent graft from cutting blades may be time consuming and/or can damage the graft during the removal process. Finally, the support block may contaminate the graft with cut debris, e.g., wood fibers, and may be too bulky to place near a patient to transfer the graft.

Embodiments disclosed herein relate to devices for fenestrating a skin graft, hereinafter referred to as a “fenestrator device” or “fenestrator,” and related methods, compositions of matter, kits, and systems. Such fenestrator devices can enable a medical professional to rapidly perforate a graft before utilizing the graft to treat a patient (e.g., before implanting the fenestrated graft). In some non-limiting aspects, compared to the mesher devices described above, the fenestrator devices described herein may be lighter in weight and/or may cool more quickly after heat sterilization.

Embodiments of fenestrator devices disclosed herein may be constructed of plastic and may be sterilized by chemical methods that do not employ heat and, thus, do not require a period of cooling before reuse. Further, embodiments of fenestrator devices disclosed herein may be disposable after a single use, obviating the need for a subsequent sterilization process. Also, the fenestrator devices disclosed herein may include fewer constituent parts or components than mesher devices and, thus, may be easier to clean and prepare before sterilization. As a result, it may be possible to produce a disposable fenestrator device for less than the cost of cleaning, wrapping, and sterilizing the mesher devices discussed above. Embodiments of fenestrator devices disclosed herein may be, for example, one tenth or less of the volume of mesher devices, enabling efficient use of limited storage space in a medical environment.

Moreover, the fenestrator devices of the present disclosure may be simpler to use than mesher devices, and their use may be simpler to teach to a medical professional. For example, unlike mesher devices, the appearance of a fenestrator device disclosed herein may intuitively suggest how it is to be used. Additionally, embodiments of the fenestrator devices disclosed herein may produce smaller perforations in graft material than the linear incisions created by the mesher devices, thereby conserving the strength of the graft while allowing sufficient drainage of exudates from the recipient site. The smaller perforations may also produce an aesthetic result that is pleasing to the patient after healing. Furthermore, because the devices can be less expensive and/or be disposable, the devices can come in a variety of sizes and/or be capable of producing different sized and/or shaped perforations. In contrast, mesher devices with their high cost do not as easily allow for efficient and lower cost flexibility and ease of use.

Non-limiting, illustrative embodiments are now described. Other embodiments may be used in addition or instead. Details which may be apparent or unnecessary may be omitted to save space or for a more effective presentation. Some embodiments may be practiced with additional components or steps and/or without all of the components or steps which are described.

To assist in the description of the devices, systems, and methods described below with reference to the figures, the following coordinate terms are used, consistent with the coordinate axes illustrated. A “longitudinal axis” is normal to a “lateral axis.” A “transverse axis” extends normal to both the longitudinal and lateral axes. In addition, as used herein, “the longitudinal direction” refers to a direction substantially parallel to the longitudinal axis; “the lateral direction” refers° to a direction substantially parallel to the lateral axis; and “the transverse direction” refers to a direction substantially parallel to the transverse axis. The terms “upper,” “lower,” “top,” “bottom,” “under side,” “upper side” and the like, which may be used to describe fenestrator devices and related components in the discussion below, are used in reference to the illustrated orientation of embodiments.

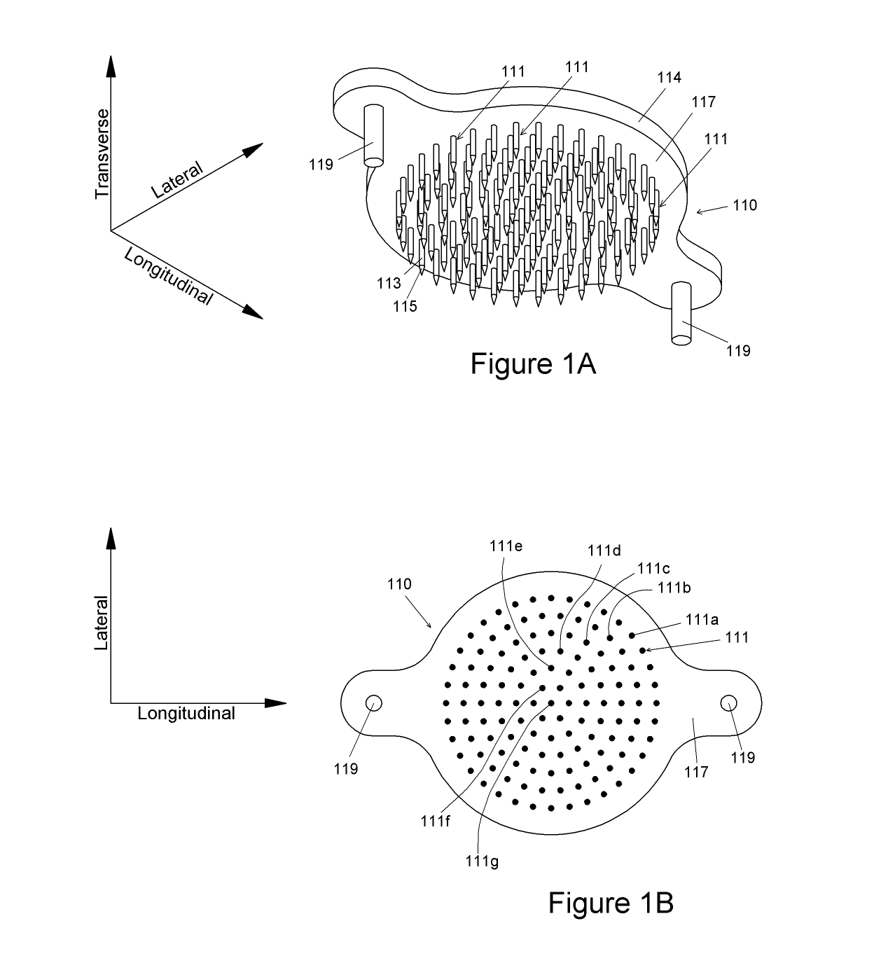

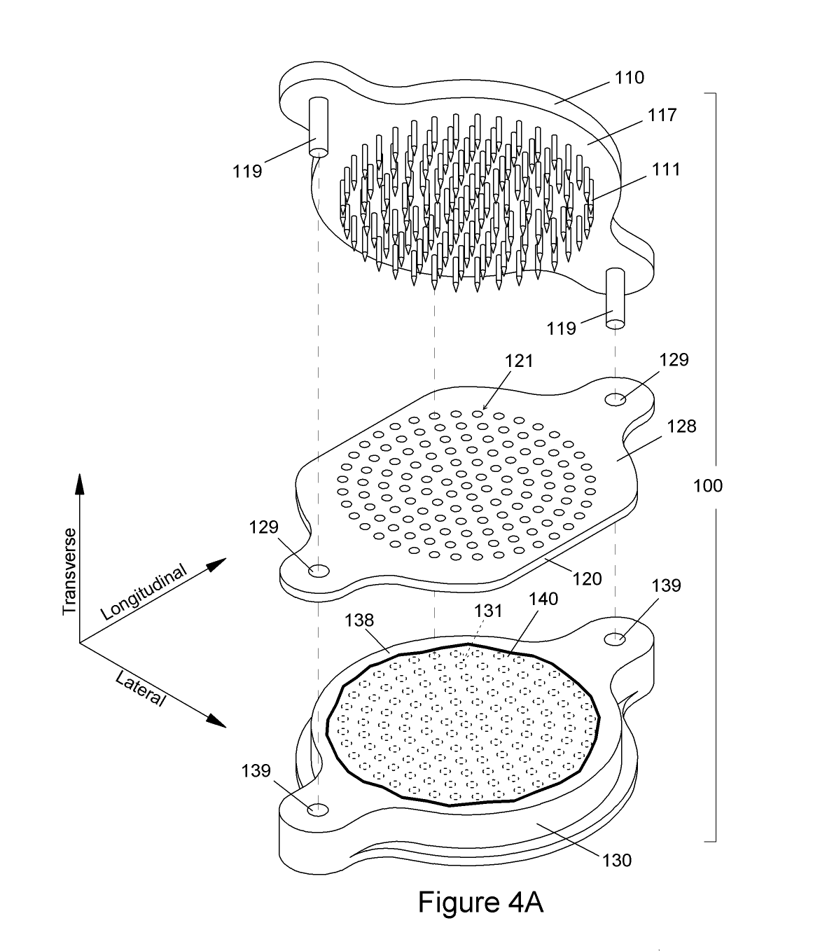

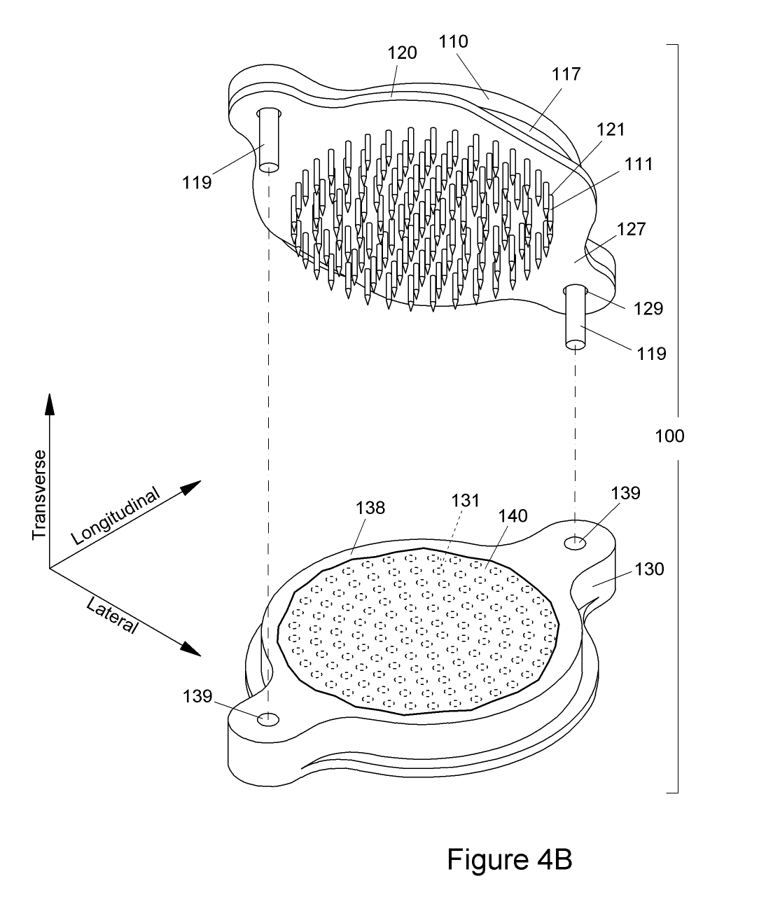

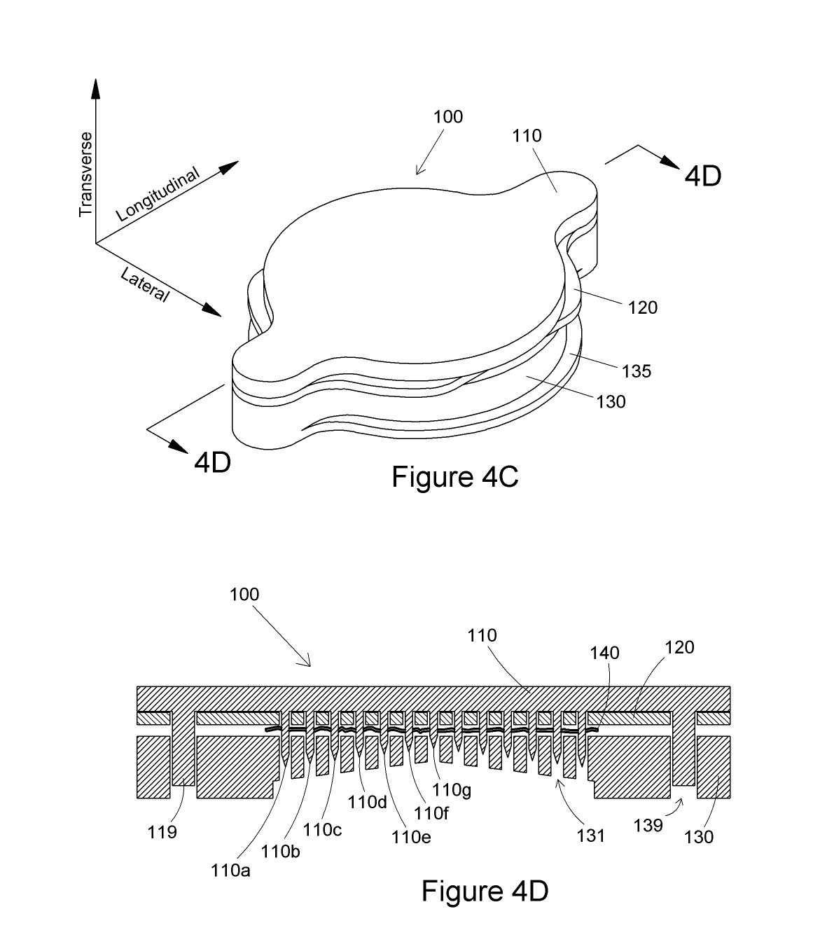

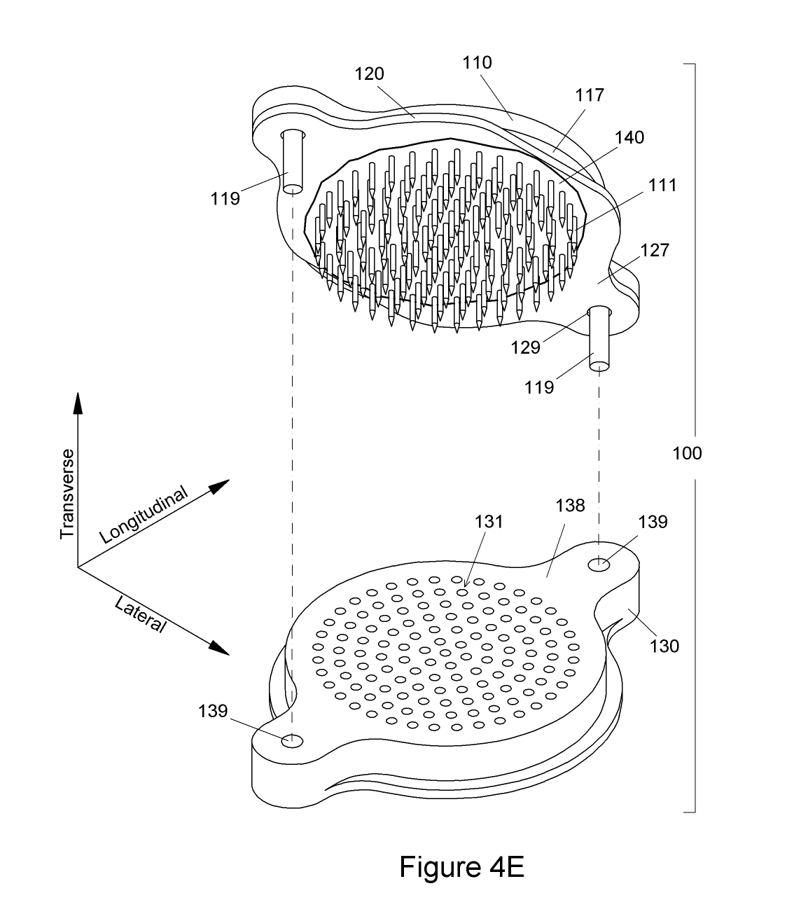

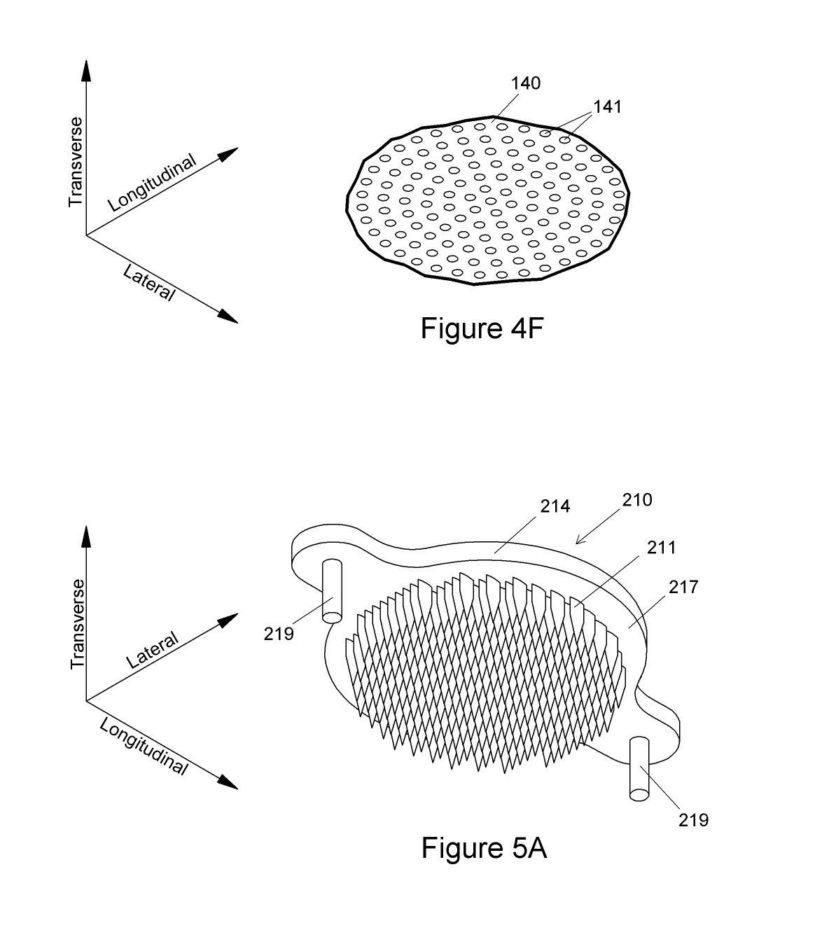

FIG. 1A schematically illustrates a bottom perspective view of an example of a perforation component 110. FIG. 1B schematically illustrates a bottom plan view of the perforation component 110 of FIG. 1A. As discussed below, the perforation component 110 can form part of a fenestration device 100 (as shown in FIG. 4A).

As illustrated in FIGS. 1A and 1B, the perforation component 110 includes a plurality of perforating elements 111 extending in the transverse direction away from a bottom surface 117 of a body 114. The perforating elements 111 can comprise various structures, including, for example, spikes, lances, pins, spears, nails, barbs, prickles, quills, thorns, bristles, blades, or any other structures capable of perforating a graft. As shown in FIG. 1A, in some embodiments, the perforating elements 111 can include a stem 113 that extends transversely away from the bottom surface 117 and a pointed end 115 disposed at a transverse end of the stem. In this way, the pointed end 115 may perforate a graft and the stem 113 may be advanced through the perforation to form and/or shape an aperture, hole, or opening in the graft. In some embodiments, the perforating elements 111 can be retractable such that a transverse length that extends from the bottom surface 117 can vary. For example, in some embodiments, the perforating elements 111 can be moved between a retracted state wherein the elements are housed within the body 114 and do not extend from the bottom surface 117, and an unretracted state (see FIG. 1A) wherein the elements 111 extend in a tranverse direction away from the bottom surface 117 outside of the body 114.

In some embodiments, the perforation component 110 can include a plurality of different perforating elements 111a-111g. For example, the various perforating elements 111a-111g may disposed radially, for example, in circles along the bottom surface 117 such that perforating elements 111a are disposed on the outside of all perforating elements 111, perforating elements 111b are disposed between perforating elements 111a and 111c, perforating elements 111d are disposed between perforating elements 111c and 111e, and perforating elements 111f are disposed between perforating elements 111e and perforating element 111g. As discussed in further detail below, in some embodiments, perforating elements 111a-111g may vary from one another in at least one of size, material, and shape. For example, a maximum transverse dimension may vary between perforating elements 111a-111g such that the maximum transverse dimension of perforating element 111g is less than the maximum transverse dimension of perforating elements 111a. In some embodiments, the perforating elements 111 may be patterned to produce a uniform pattern of openings, holes, or apertures in a graft to be treated. Accordingly, the shape, size, number, pattern, and/or spatial distribution of the perforating elements 111 can vary from one embodiment to another. In some embodiments, a pattern of the perforating elements 111 can be symmetrical such that the perforation component 110 may be flipped or rotated, for example, across the lateral axis without changing the distribution of perforating elements 111. While a radial pattern of perforating elements 111 is depicted in FIGS. 1A-1B, it should be understood that a variety of patterns can be utilized with different spacing, geometric shape, size of perforating element 111, etc.

In some embodiments, the perforating elements 111 are configured to produce holes or incisions having lengths of between 2 mm and 4 mm, e.g., 3 mm, in a graft. Additionally, in some embodiments, the perforating elements 111 can optionally be configured to produce holes or incisions in a graft with each hole being separated from each other hole in the longitudinal direction by between 2 mm and 4 mm, e.g., 3 mm. Further, in some embodiments, the perforating elements 111 can optionally be configured to produce holes or incisions in a graft with each hole being separated from each other hole in the lateral direction by between 1 mm and 2 mm, e.g., 1.5 mm. In some embodiments, the patterning and/or configuration of the perforation elements can be selected based on the cosmetic appearance of the resultant holes. For example, if a graft is intended for use in a cosmetically important area of a patient the perforation component 110 may include perforation elements 111 that are spaced apart from one another such that a treated graft has fewer holes than in another application.

As shown in FIG. 1A, in some embodiments, the perforation component 110 may also include one or more alignment dowels 119 extending in a transverse direction away from the bottom surface 117. As discussed in more detail below, the one or more alignment dowels 119 may be used to position and/or align the perforation component 110 relative to other components or structures of a fenestrator device. For example, in some embodiments, the alignment dowels 119 may be received within a corresponding hole, slot, or aperture such that the perforating elements 111 are disposed in a desired position relative to a skin graft and/or to a base component. While the depicted embodiment utilizes and refers to “alignment dowels,” it should be understood that any other suitable mechanism can be used to properly align the perforation component 110 with other components, structures, etc (e.g., a support plate as described below). For example, the perforation component 110 can have a hole or aperture to receive a dowel or other protrusion from some other structure in order to align the perforation component 110 with the other structure. In some aspects, both the perforation component 110 and the other structure can have one or more protrusions/dowels and one or more holes/receiving parts that can line up with the corresponding elements on the other device in order to align the perforation component 110 with the other structure, whatever that structure may be.

With continued reference to FIGS. 1A and 1B, the body 114 of the perforation component 110 may form or comprise a variety of shapes (e.g., any geometric shape such as square, rectangular, round, circular, oval, curvilinear, etc.). For example, as illustrated, the body 114 may be substantially round or curvilinear and/or may have a maximum transverse dimension that is less than a maximum longitudinal dimension and/or a maximum lateral dimension. In other embodiments, the body 114 may include one or more linear edges. In some embodiments, the size and shape of the body 114 may be selected such that the perforation component 110 is easy to handle and manipulate by a medical professional. For example, the perforation component may have a maximum longitudinal dimension of between 50 mm and 250 mm, a maximum lateral dimension of between 50 mm and 250 mm, and a maximum transverse dimension of between 1 mm and 50 mm. In some embodiments, the bottom surface 117 can have a surface area of between 1,500 mm² and 200,000 mm². In some aspects, the bottom surface 117 can have a surface area of between 1,500 mm² and 62,500 mm². In some embodiments, the perforation component 110 and the support plate 120 and base 130 (discussed below) can be similarly sized and/or shaped.

In some embodiments, the perforation component 110 may be formed of or may include one or more non-metallic materials, for example, a plastic material (e.g., any of a wide range of synthetic or semi-synthetic organic solids such as thermoplastics and thermosetting polymers, for example), a ceramic material, a rubber, other carbon fiber or carbon materials, or any other suitable material. In other embodiments, the perforation component 110 can include one or more metals or alloys, for example. In some embodiments, the perforating elements 111 may include a different material than the body 114, and in other embodiments, the perforating elements 111 and the body 114 may be formed of the same material(s). For example, the perforating elements 111 can be made of or may include, at least in part, any suitable material, including for example, a plastics material, a metal or alloy, a ceramic material, a rubber, etc.

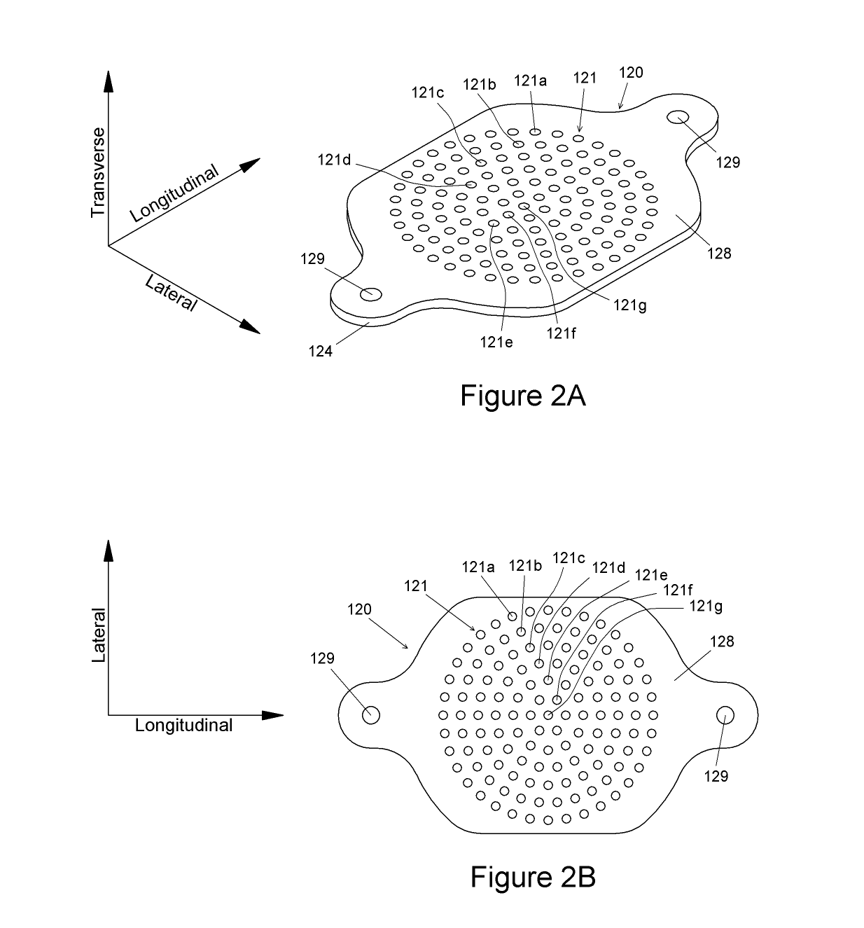

FIG. 2A schematically illustrates a top perspective view of a non-limiting example of a support plate 120. FIG. 2B schematically illustrates a top plan view of a non-limiting example of the support plate 120 of FIG. 2A. As illustrated in FIGS. 2A and 2B, the support plate 120 includes a plurality of holes, openings, or apertures 121 extending through a body 124 between a top surface 128 and a bottom surface (shown as 127 in FIG. 4B) of the support plate. The quantity and pattern of the holes 121 can match the quantity and pattern of the perforating elements 111 of the perforation component 110 of FIGS. 1A and 1B, for example. Additionally, the holes 121 may be sized, shaped, and/or arranged such that the perforating elements 111 can extend at least partially therethrough. In some embodiments, the holes 121 may be sized larger than the perforating elements 111 so as to prevent dulling contact between the perforating elements 111 and the holes 121 when the perforating elements 111 are extended at least partially through the holes. In this way, the perforating elements 111 can be repetitively extended into and out of the holes 121 without being dulled by contact. Accordingly, in some embodiments, the plurality of holes 121 can include holes 121a-121g that may vary in at least one of size, shape, or pattern. For example, a diameter of hole 121g may be less than, or greater than, a diameter of holes 121a.

In some embodiments, the support plate 120 may include one or more alignment apertures 129. The number, size, and shape of the alignment apertures 129 can correspond to the number, size, and shape of the alignment dowels 119 of the perforation component 110 of FIGS. 1A and 1B, for example. In this way, in some embodiments, the alignment apertures 129 can receive the alignment dowels 119 therethrough such that the perforation component 110 may be properly aligned or positioned relative to the support plate 120. Thus, in some embodiments, the alignment apertures 129 can serve to align the perforating elements 111 relative to the holes 121 such that the perforating elements 111 extend at least partially into the holes 121 when the bottom surface 117 of the perforation component 110 is disposed over the top surface 128 of the support plate 120.

It should be understood that any of various suitable approaches can be utilized to align the support plate 120 with the perforation component 110, for example, such that the desired perforating elements 111 and the correspondingly desired holes 121 are in proper alignment. For example, the support plate 120 can include the protruding element such as the “dowel” rather than the alignment apertures. In some aspects, the support plate as well as the perforation component may include one or more apertures and one or more protruding elements, etc. In some aspects, other alignment strategies may be utilized which result in at least some of the perforating elements 111 aligning with at least some of the holes 121.

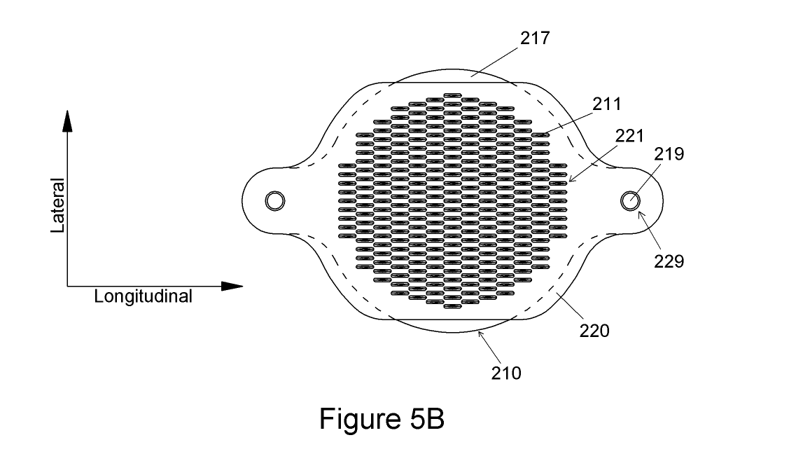

With continued reference to FIGS. 2A and 2B, the body 124 of support plate 120 may form a variety of shapes (e.g., any geometric shape such as square, rectangular, round, circular, oval, curvilinear, etc.). For example, as illustrated, in some embodiments, the body 124 may be oblong with round ends and straight lateral sides along the mid-portion of the body. As discussed in further detail with respect to FIG. 5B, in some embodiments, a cross-sectional area of the support plate 120 may be larger than a cross-sectional area of the perforation component 110 to allow a medical professional to easily separate the support plate from the perforation component when the perforating elements 111 are disposed through the holes 121. In some embodiments, the support plate 120 may be formed of one or more non-metallic materials, for example, a plastics material, a ceramic material, other carbon based or carbon fiber materials, rubbers, and the like. In other embodiments, the support plate 120 can include one or more metals and/or alloys, for example.

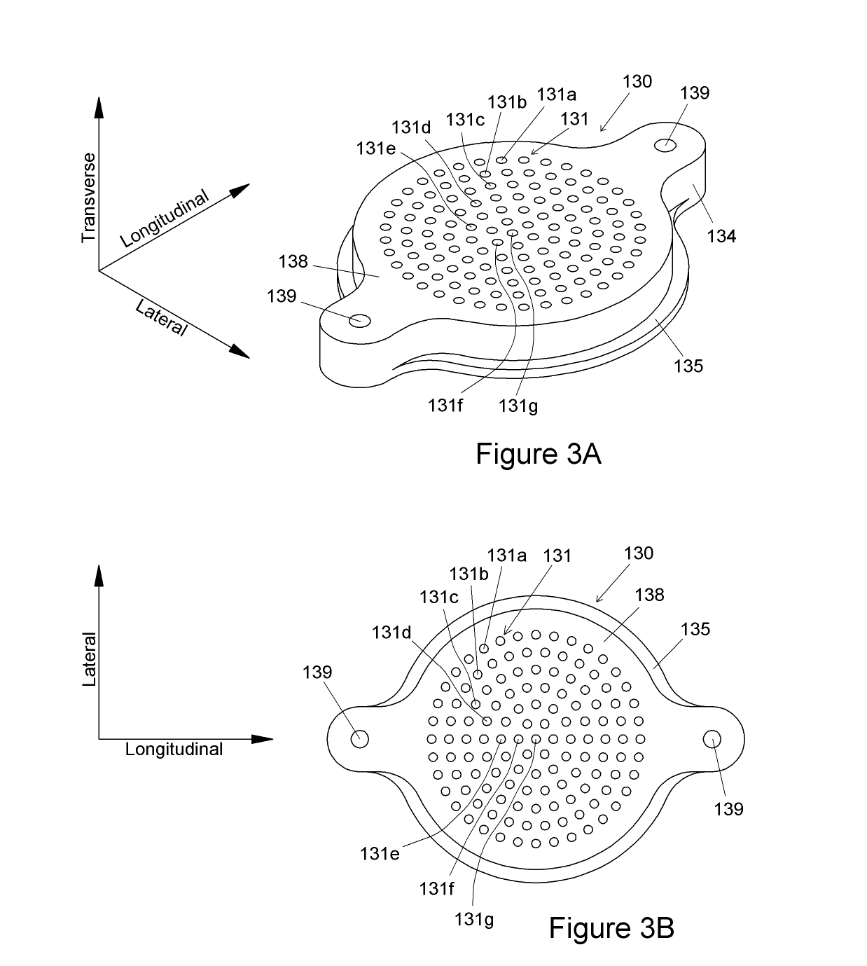

FIG. 3A schematically illustrates a top perspective view of a non-limiting example of a base 130. FIG. 3B schematically illustrates a top plan view of a non-limiting example of the base 130 of FIG. 3A. As illustrated in FIGS. 3A and 3B, the base 130 includes a plurality of receptacles or receiving spaces 131 extending from a top surface 138 toward a bottom surface (not shown) of the base. The quantity and pattern of the receptacles 131 can match the quantity and pattern of the perforating elements 111 of the perforation component 110 of FIGS. 1A and 1B, for example. Moreover, the quantity and pattern of the receptacles 131 can match the quantity and pattern of the holes 121 of the support plate 120 of FIGS. 2A and 2B, for example. Additionally, the receptacles 131 may be sized, shaped, and/or arranged such that the perforating elements 111 can extend at least partially into the receptacles 131. In some embodiments, the receptacles 131 may be sized larger than the perforating elements 111 so as to prevent dulling contact between the perforating elements 111 and the receptacles 131 when the perforating elements 111 are extended at least partially into the receptacles 131. Accordingly, in some embodiments, the plurality of receptacles 131 can include receptacles 131a-131g that may vary in at least one of size, shape, or pattern. For example, a diameter of receptacles 131g may be less than, or greater than a diameter of receptacles 131a. In some aspects, the receptacles 131 can extend completely through the base 130. In some aspects, such receptacles 131 can facilitate cleaning, since material might more easily be removed from the receptacles 131 as part of a cleaning or sterilization process. In other aspects, the receptacles 131 may not extend completely through the base 130, for example.

In some embodiments, the base 130 may include one or more alignment receptacles 139. The number, size, and shape of the alignment receptacles 139 can correspond to the number, size, and shape of the alignment dowels 119 of the perforation component 110 of FIGS. 1A and 1B, and to the number, size, and shape of the alignment apertures 129 of the support plate 120 of FIGS. 2A and 2B. In this way, the alignment receptacles 139 can at least partially receive the alignment dowels 119 such that the perforation component 110 and support plate 120 may each be properly aligned or positioned relative to the base 130. That is to say, the perforating elements 111, holes 121, and receptacles 131 can be coaxially aligned such that the perforating elements 111 can extend through the support plate 120 and into the base 130. As noted above, the depicted approach for aligning the base 130 is just one example of an alignment approach. It should be understood that in some aspects, the base 130 can have the protruding elements rather than the alignment receptacles 139. In other aspects, the base 130 can have both protruding elements (e.g., dowels) and receptacles 139. In other aspects, any other suitable alignment mechanism (e.g., notches, ridges, channels, bumps, etc.) can be utilized, for example, to align the base 130 with one or more other structures, such as the perforation component 110 and/or the support plate 120.

With continued reference to FIGS. 3A and 3B, the body 134 of the base 130 may form a variety of shapes (e.g., any geometric shape such as square, rectangular, round, circular, oval, curvilinear, etc.). For example, as illustrated, the body 134 may be substantially round or curvilinear and/or may have a maximum transverse dimension that is less than a maximum longitudinal dimension and/or lateral dimension. In other embodiments, the body 134 may include one or more linear edges. In some embodiments, the size and shape of the body 134 may be selected such that the base 130 is easy to handle and manipulate by a medical professional. For example, the body 134 optionally may include one or more flanges 135 or gripping surfaces configured to provide a force bearing surface for a medical professional to manually grip the body 134. In this way, the flange 135 may be used to separate the base 130 from other components, for example, the perforation component 110 and/or support plate 120. In some embodiments, the size and shape of the body 134 may be selected such that a top surface 138 of the base 130 is configured to support a graft disposed at least partially over the top surface. In some aspects, the base 130 can have a size or dimension that is at least partially the same as, larger than, or smaller than the size of the perforation component 110 and/or the support plate 120. In some embodiments, the base 130 may be formed of one or more non-metallic materials, for example, a plastics material, a ceramic material, other carbon based or carbon fiber materials, rubbers, and the like. In other embodiments, the base 130 can include one or more metals and/or alloys.

FIG. 4A schematically illustrates an exploded view of an example of a fenestrator device 100 including the perforation component 110 of FIGS. 1A and 1B, the support plate 120 of FIGS. 2A and 2B, the base 130 of FIGS. 3A and 3B, along with an example graft 140. FIG. 4B schematically illustrates a partially exploded view of the fenestrator device of FIG. 4A showing the perforation elements 111 of the perforation component 110 extending through the support plate 120, along with an example graft 140. FIG. 4C schematically illustrates the fenestrator device 100 of FIG. 4A with the perforation component 110 and the support plate 120 disposed directly over the base 130. FIG. 4D schematically illustrates a cross-sectional view of the fenestrator device 100 of FIG. 4C taken along the transverse line 4D-4D.

As shown in FIG. 4B, the alignment dowels 119 of the perforation component 110 are extended through the alignment apertures 129 of the support plate 120 such that the perforating elements 111 are aligned with, and extend through, the holes 121. In some embodiments, to facilitate the relative alignment of the perforation component 110 and the support plate 120, the transverse length of the alignment dowels 119 may be greater than a maximum transverse length of the perforating elements 111 such that the alignment dowels 119 may be aligned with the alignment apertures 129 prior to extending the perforating elements 111 through the holes 121. Although schematically illustrated in FIGS. 4A-4E as separate components, in some embodiments the perforation component 110 and the base 130 can be joined together by one or more hinge components. In such embodiments, the hinge component can be configured such that the perforation component 110 can be aligned with the support plate 120 and/or the base 130 by moving the perforation component 110 relative to the base 130 about the hinge component. In this way, the perforation component 110 and the base 130 can form halves of a “clam shell” that are movable relative to one another.

Once the perforation component 110 and the support plate 120 are properly aligned, the top surface 128 (shown in FIG. 2A) of the support plate 120 may be positioned against the bottom surface 117 of the perforation component 110 such that the perforation component 110 and the support plate 120 can be manipulated together with the perforating elements 111 extending transversely downward. It should be noted that in some aspects the support plate 120 can be aligned first with the base 130, while in other embodiments, it can be aligned first with the perforation component 110 as described above. Further, in some embodiments, a fenestrator device can include the perforation component 110 and the base 130 without including the support plate 120.

With continued reference to FIG. 4B, the graft 140 schematically illustrated may include any suitable skin graft and/or skin substitute material that may be used by a medical professional to treat a skin defect and/or to stimulate wound healing. An example of a suitable graft 140 is an APLIGRAF® bio-engineered graft that is commercially available from Organogenesis of Inc. of Canton, Mass. In some embodiments, the graft 140 may be provided with at least some of the components of the fenestrator device 100 (e.g., the perforation component 110, the support plate 120, and/or the base 130) in a kit such that a medical professional may be provided with all of the materials necessary to perforate a graft and treat a patient in one package or kit (e.g., a packaged and pre-sterilized kit). Other graft materials can be included in kits, and the APLIGRAF® is one non-limiting example of a graft material that can be used.

As illustrated in FIGS. 4A and 4B, the graft 140 may be disposed over the top surface 138 of the base 130 such that the graft at least partially covers at least some of the receptacles 131. In this way, the graft 140 may be perforated by the perforating elements 111 when the alignment dowels 119 are received within the alignment receptacles 139 of the base 130 and the perforation component 110 is pushed transversely downward until the bottom surface 127 of the support plate 120 contacts the graft 140 as illustrated in FIGS. 4B and 4C.

FIG. 4C illustrates a non-limiting example the fenestrator device 100 of FIG. 4B with the perforating elements 111 (not shown) of the perforation component 110 disposed at least partially within the receptacles 131 of the base 130. The graft 140 is not shown, but is “sandwiched” between the support plate 120 and the base 130. Also shown is the gripping surface 135, which will facilitate separation of the various pieces so that the fenestrated graft can be removed and used with a patient. FIG. 4D further illustrates the various components in contact as illustrated in FIG. 4C.

Turning now to FIG. 4D, when the alignment dowels 119 are received within the alignment receptacles 139 and the support plate 120 is in contact with the graft 140, the perforating elements 111 may extend through the graft 140 and extend at least partially into the receptacles 131 of the base 130. In some embodiments, when the support plate 120 is in contact with the graft 140, a user may detect a clear end point of transverse motion indicative of the graft 140 being penetrated by the perforating elements 111a-111g. As discussed above and as illustrated in FIG. 4D, in some embodiments, the perforation component 110 can include different perforating elements 111a-111g having different transverse lengths. In this way, the perforating elements 111 may be configured to progressively contact the graft 140 (e.g., from the longer outer perforating elements 111a to the shorter inner perforating element 111g), thereby reducing the effort or force required by a user to perforate the graft 140 with the perforating elements 111. In other embodiments, the perforating elements 111 can have the same lengths, if desired.

In some embodiments, the body 134 (see FIG. 3A) of the base 130 may be sufficiently sized and shaped so as to at least partially shield the perforating elements 111 from a user. For example, as illustrated, the receptacles 131 may be disposed within a center portion of the body 134 and may not extend through the bottom surface of the base such that a user may not inadvertently contact the perforating elements 111 when they are extended into the receptacles during usage. In other embodiments, the perforating elements can extend completely at least partially through the bottom surface of the base 130.

Turning now to FIG. 4E, the fenestrator device 100 is shown with the perforation component 110 and support plate 120 removed from the base 130 (e.g., removed from the position illustrated in FIGS. 4C and 4D). As was shown in FIG. 4C, in some embodiments, the support plate 120 can extend beyond the lateral sides of the base 130 enabling a user (e.g., a medical professional) to easily grip the overhanging support plate and separate the support plate 120 and perforation component 110 from the base 130 with one or more gloved hands. As depicted in FIG. 4E, the perforation component 110 and the support plate 120 have been removed from the base 130 together. It should be understood that this is merely one example and that in some embodiments the perforation component 110 can be removed while the support plate 120 remains in contact with the tissue 140 and the base 130, for example.

As shown in FIG. 4E, in some embodiments, the graft 140 may be frictionally attached to the perforating elements 111 and/or support plate 120 by design. The graft 140 may then be separated from the perforation component 110 by separating the support plate 120 from the perforation component 110. In some embodiments, a difference in cross-sectional shape and/or size between the perforation component 110 and the support plate 120 may facilitate the removal of the graft 140 from the perforating elements 111. That is to say, the support plate 120 may extend laterally beyond the lateral sides of the perforation component 110. Such a configuration may provide means for facilitating separation of the components from one another when the graft 140 is disposed therebetween. For example, the extended surface of the support plate 120 may be grasped in one hand, the exposed surface 117 of the perforation component 110 may be grasped in another hand, and opposing forces can be applied by each hand to separate the support plate 120 from the perforation component 110.

In some embodiments, because the perforating elements 111 may include perforating elements 111a-111g having progressively different transverse lengths, the frictional attachment of the graft 140 to the perforating elements 111 can also be progressively varied. Accordingly, this feature may reduce the risk of a sudden release of the graft 140 from the perforating elements 111 and a corresponding loss of control of the graft 140 as it separates from the perforation component 110. The graft 140 may then be easily transported on the support plate 120 and approximated to a recipient patient for use in treatment. As discussed above, in some embodiments, the perforating elements 111 can be retracted within the body 114 of the perforation component 110. In such embodiments, the graft 140 can be separated from the perforating elements 111 by retracting the perforating elements. Again, in some aspects, the perforation component 110 can be removed while the support plate 120 and base 130 remain in contact (e.g., thereby supporting the graft 140) with the graft 140 in between. Upon removal of the perforation component 110, the graft 140 can be transported for use, if desired, with either or both of the support plate 120 and the base 130.

The fenestrator device 100 of FIGS. 4A-4E and/or one or more of its components may be constructed of one or more durable materials, for example, stainless steel and/or aluminum, that are capable of being repeatedly sterilized for re-use. Any other metal or alloy, alone or combined, can be used as well. Additionally or alternatively, the fenestrator device 100 and/or one or more of its components may also be constructed of one or more other durable materials, for example, one or more plastics, ceramics, rubbers, etc. In some embodiments the fenestrator devices and/or their components can be capable of being repeatedly sterilized for re-use. Also, in some embodiments the fenestrator devices can be less costly, e.g., such that they may be disposed after a single use or after 2-20 uses before being disposed of. In some embodiments, the fenestrator device 100 or one or more of its components can be constructed from a biodegradable or compostable material such that a single use of the device or a component has minimal environmental effects.

FIG. 4F schematically illustrates the graft 140 of FIGS. 4B, 4D, and 4E after the graft 140 has been perforated by the perforating elements 111. As shown, the graft 140 includes a plurality of openings, holes, or apertures 141. In some embodiments, the size, shape, quantity, and spatial distribution or patterning of the holes 141 can mirror the size, shape, quantity, and spatial distribution or patterning of the perforating elements 111 of the perforation component 110. The holes 141 may desirably allow wound exudates to pass through the graft 140 and/or may facilitate stretching of the graft 140 during implantation. Thus, the graft 140 may be preferable to a similarly sized and shaped graft that does not include holes 141. While the holes 141 as depicted are round, it should be understood and noted that the devices herein can be designed to create any desired size and shape of perforation in the material that is been fenestrated (e.g., natural or synthetic tissue or material).

Turning now to FIG. 5A, another non-limiting example of an embodiment of a perforation component 210 for use in a fenestration device is schematically illustrated. Similar to the perforation component 110 discussed above, the perforation component 210 includes a plurality of perforating elements 211 extending transversely away from a bottom surface 217 of a body 214. Further, in some embodiments, the perforation component 210 includes a pair of alignment dowels 219 that may be used to align or position the perforation component 210 relative to one or more additional components of a fenestration device and/or relative to a skin graft. However, in contrast to the perforation component 110 described above, the perforating elements 211 are schematically illustrated as elongated blades instead of rounder perforating elements having a stem 113 and a pointed end 115 as shown in FIG. 1A. Additionally, the perforating elements 211 are schematically illustrated as each having the same maximum transverse dimension between a tip of the blade and the bottom surface 217.

As shown in FIG. 5B, in some embodiments, the perforation component 210 can be used in conjunction with a support plate 220 having a plurality of elongated holes 221 sized and shaped so as to receive the perforating elements 211. In some embodiments, the perforating elements 211 can be aligned with the holes 221 by extending the alignment dowels 219 at least partially into alignment apertures 229 formed in the support plate 220. In this way, the perforating elements 211 can be configured to perforate a graft resulting in elongated slits or cuts as opposed to more round apertures or holes. In some embodiments, such elongated slits or cuts may be utilized to provide for greater flexibility of the graft in one direction than in another direction. For example, slits may be configured to provide for greater flexibility of the graft in the lateral direction than in the longitudinal direction. Conversely, slits may be configured to provide for greater flexibility of the graft in the longitudinal direction than in the lateral direction. In other embodiments, slits can be configured such that the graft is similarly flexible in both the lateral and longitudinal directions. Additionally, such elongated slits or cuts may result in a different aesthetic appearance of the graft region after implantation in a patient. As also shown in FIG. 5B, in some embodiments, the perforation component 210 can have a maximum lateral dimension that is greater than a maximum lateral dimension of the support plate 220. In this way, at least a portion of the bottom surface 217 of the perforation component 210 may extend beyond the support plate 220 and thereby provide a contact surface for a user to separate the perforation component 210 from the support plate 220.

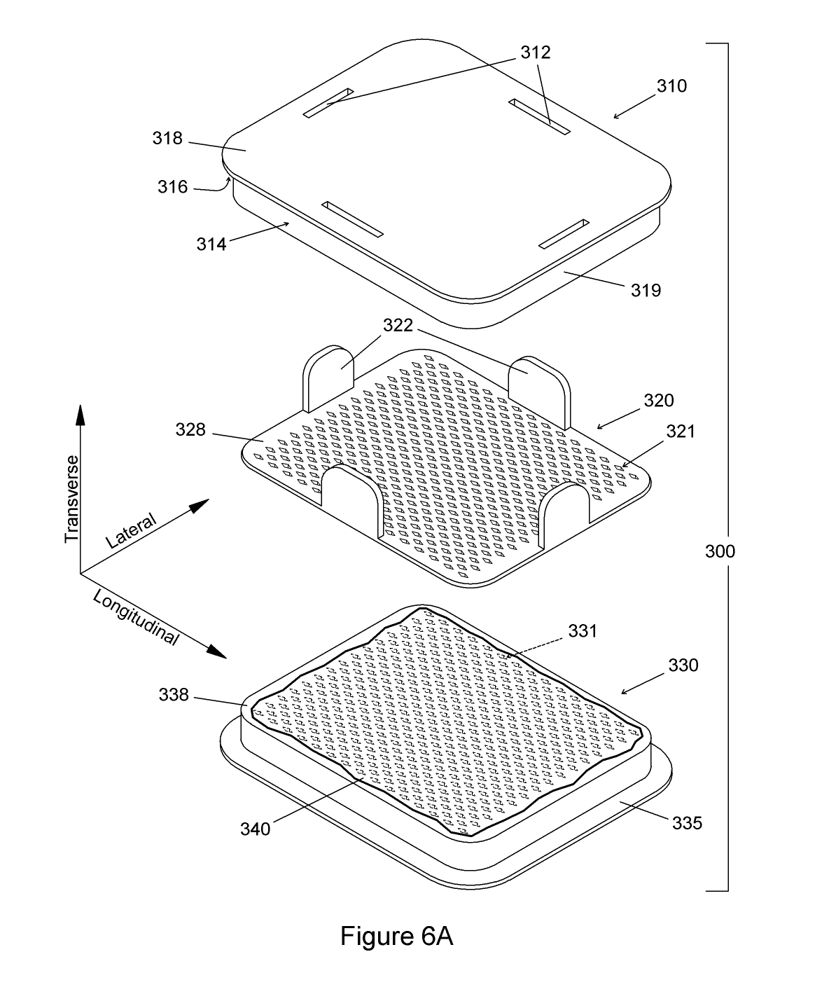

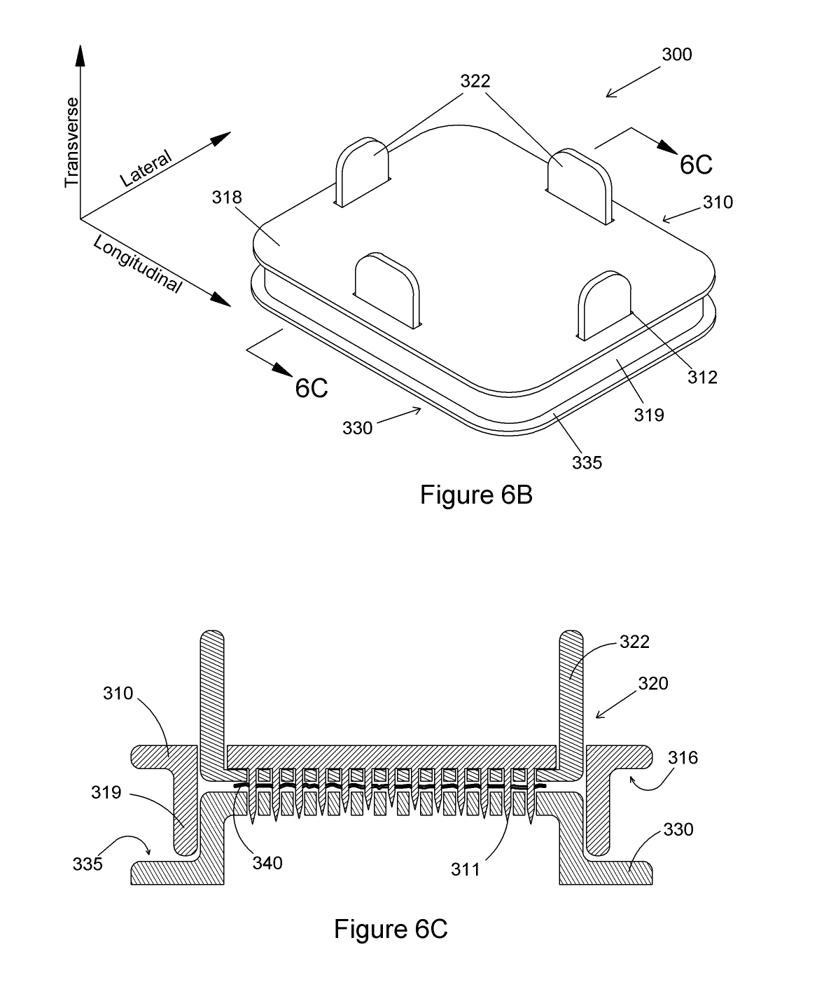

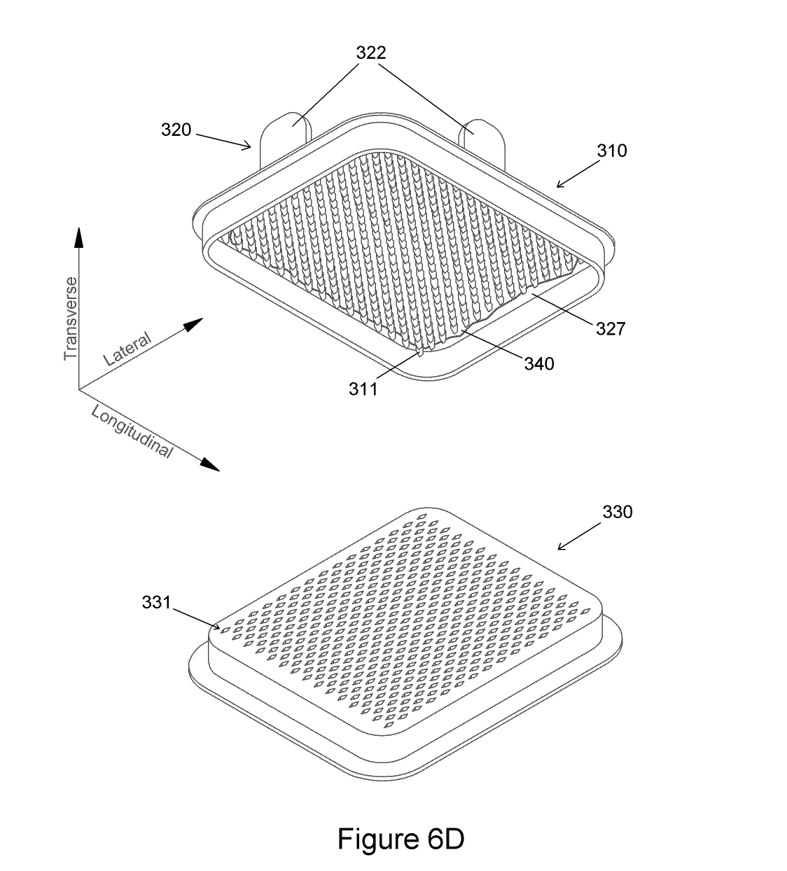

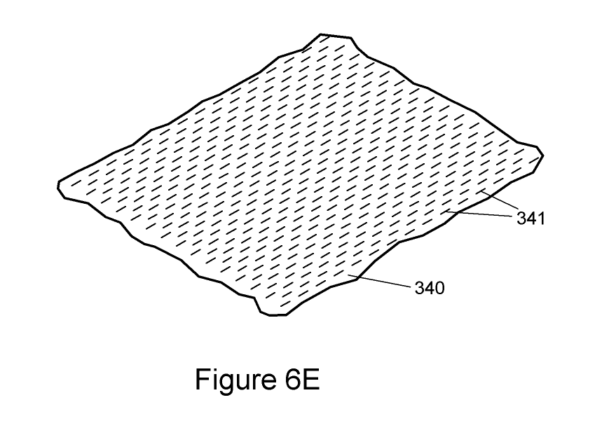

Turning now to FIGS. 6A-6D, another embodiment of a fenestration device 300 is schematically depicted. Similar to the fenestration device 100 of FIGS. 4A-4F, the fenestration device 300 can include a perforation component 310, a support plate 320, and a base 330. In some embodiments, the perforation component 310 can include one or more slots 312 extending transversely through a body 314 between a top surface 318 and a bottom surface (not shown). Additionally, the support plate 320 can include one or more tabs 322 extending transversely away from a top surface 328. The slots 312 can be sized, shaped, and arranged so as to receive the tabs 322. In this way, the slots 312 and tabs 322 can act to align the support plate 320 relative to the perforation component 310. It should be noted that the inclusion of the slots 312 and/or the tabs 322 can be switched completely or partially such that the support plate 320 has some or all of the tabs and/or perforation component 310 has some or all of the tabs 322, for example, or such that one or more tabs 322 and one or more slots 312 are included on both the perforation component 310 and the support plate 320. It should be understood that for this embodiment (or other embodiments described herein) other alignment approaches can be utilized which result in alignment of the components so that the perforating elements 311 (FIG. 6C) can align with a desired number of holes 321 or receptacles 331 of the device.

In some embodiments, the perforation component 310 can include a sleeve (rim, lip, etc.) 319 that extends transversely away from the top surface 318 at least partially along one or more edges of the perforation component. In this way, the sleeve 319 may at least partially shield perforating elements 311 of the perforation component 310 from a lateral exposure to a user, e.g., a medical professional. As shown in FIGS. 6B and 6C, in some embodiments, the sleeve 319 may also be sized and shaped so as to align the perforation component 310 relative to the support plate 320 and/or the base 330. For example, the sleeve 319 can be sized and shaped to slide over a portion of the base 330 and may be urged transversely downward such that the sleeve abuts a flange 335 of the base. In this way, the sleeve 319 may be used, in some embodiments, to align the perforating elements 311 of the perforation component 310 with receptacles 331 of the base 330. In some embodiments (not shown), the sleeve 319 can be utilized without the tabs or slots, and the sleeve can align the various components for use.

As illustrated in FIG. 6A, the holes 321 of the support plate 320 and the receptacles 331 of the base 330 may be similarly sized and shaped. For example, in some embodiments, the holes 321 and the receptacles 331 can be polygonal (e.g., diamond shaped). The holes 321 can be in any other shape as described herein as well (e.g., round, oval, square, rectangular, curvilinear, triangular, etc.). As discussed above with reference to the fenestrator device 100 disclosed herein, the holes 321 and receptacles 331 can be sized and shaped such that the perforating elements 311 can extend therethrough and/or at least partially therein without being dulled by contact. In some embodiments the holes 321 and/or the receptacles 321 can have a size that minimizes, reduces or avoids the perforated material (tissue, skin, etc.), from being pushed into the hole 321 and/or receptacle 331. For example, this can be accomplished by minimizing or eliminating space around the perforating elements 311 when the perforating elements 311 enter into or through the holes 321 and/or receptacles 331. For example, a minimal clearance space between the perforating elements 311 and the holes 321 and/or receptacles 331 can be utilized for this purpose, if desired. This can minimize, reduce or avoid the perforated material (e.g., the graft 340) being caught, attached or stuck to the base 330, the support plate 320 and/or the perforating component 310, for example, which can allow for easier use of the perforated material and/or avoid and reduce damage such as tearing or ripping of the material, for example.

As shown in FIGS. 6B-6D, in some embodiments, the tabs 322 can extend transversely beyond the top surface 318 of the perforation component 310 when the tabs 322 are received within the slots 312. This configuration may be advantageous for separating the support plate 320 from the perforation component 310 after perforating the graft 340. For example, in some embodiments, a user may grip the flange 316 of the perforation component 310 and/or the flange 335 of the base 330 to separate the perforation component 310 from the base 330. After such disengagement between the perforation component 310 and the base 330, the support plate 320 may remain engaged with the perforation component 310 due to the frictional engagement between the graft 340 and the perforating elements 311. That is to say, the perforated graft 340 may act to hold the support plate 320 to the perforation component 310, in some embodiments.

In some embodiments, the upwardly extending tabs 322 can facilitate the separation of the support plate 320 and the graft 340 from the perforating elements 311. For example, a user may place the perforation component 310 on a fixed surface, e.g., a table top, such that the top surface 318 faces the fixed surface. In some embodiments, the user may then press downward on the flange 316 of the perforation component 310 such that the slots 312 slide over the tabs 322 and the perforating elements 311 are separated from the graft 340. With the graft 340 resting atop the bottom surface 327 of the support plate 320, the user may remove and/or transport the fenestrated graft for use in treating a patient. Although FIGS. 6B-6D depict the support plate 320 being removed from the base 330 with the perforation component 310, in some embodiments, the perforation component 310 can be separated from the base 330 without the support plate 320. For example, the perforation component 310 may be removed from the base 330 without separating the support plate 320 from the base such that the base and the support plate each contact the graft 340 therebetween even after the perforation component 310 has been removed.

The fenestrator devices disclosed herein can be utilized in accordance with various methods. For example, in some embodiments, a method of use can include placing the base 330 on a fixed surface using sterile technique. A graft 340 may then be evenly spread over the top surface 338 of the base 330 such that the graft 340 at least partially covers at least some of the receptacles 331. In some embodiments, the tabs 322 of the support plate 320 may be guided through the corresponding slots 312 of the perforation component 310 such that the perforating elements 311 extend at least partially through the holes 321 of the support plate. The perforation component 310 may then be aligned with the base 330 by sliding the sleeve 319 over at least a portion of the base 330 such that the perforating elements 311, holes 321, and receptacles 331 are coaxially aligned. From this position, in some embodiments, the perforation component 310 may be manually urged or moved toward the base 330 until the perforating elements 311 have perforated or penetrated the graft 340. Such perforation or penetration can be indicated by an abutment between the flange 335 of the base 330 and the sleeve 319. Additionally or alternatively, such perforation or penetration can be indicated by an abutment between the bottom surface of the support plate 320 and the graft 340. In some embodiments, the flange 316 of the perforation component 310 and/or the flange 335 of the base 330 may be utilized to separate the perforation component 310 and support plate 320 from the base 330. Because the graft 340 may deform and frictionally engage the perforating elements 311, the graft may also be separated from the base 330 in this way. Accordingly, in some embodiments, the graft 340 may be separated from the perforating elements 311 by separating the support plate 320 from the perforation component 310 as discussed above. The support plate 320 and the graft 340 may then be transported to a treatment site where the graft 340 may be separated from the support plate 320 and utilized for a treatment.

FIG. 6E schematically illustrates the graft 340 of FIGS. 6A, 6C, and 6D after the graft has been perforated by the perforating elements 311. As shown, the graft 340 includes a plurality of openings, holes, apertures, or slits 341. In some embodiments, the size, shape, quantity, and spatial distribution or patterning of the slits 341 can mirror the size, shape, quantity, and spatial distribution or patterning of the perforating elements 311 of the perforation component 310. The holes 341 may desirably allow wound exudates to pass through the graft 340 and/or may facilitate stretching of the graft 340 during implantation.

Some embodiments relate to kits that can include, for example, one or more of the devices, components, elements, materials, etc. disclosed herein. In some embodiments, instructions for using any of the fenestrator devices disclosed herein or substituent components may be provided in a kit along with one or more grafts or products for grafting or skin or wound healing, for example. The grafts or products can include natural skin (e.g., without limitation foreskin from a human or other animal). The graft can be, for example, autologous, isogeneic, allogeneic, xenogeneic, etc. The grafts can be synthetic, for example, an artificial skin and/or another material comprised of “plastics” (e.g., polymers, etc. of various types), metallic, ceramic, etc. (prosthetic implants). In some embodiments, instructions for using the fenestrator devices or related components can be represented by indicia that are placed on one or more components of the device. For example, a perforation component can include text and/or illustrations displaying a sequence of steps for a user. In some embodiments, a fenestrator device can be provided without a kit and can include indicia representing instructions for using the device.

The fenestrator devices disclosed herein can be simple to use and compact. In some embodiments, such fenestrator devices can be easily cleaned and sterilized for reuse (e.g., by heat or chemical sterilization methods). In some embodiments, usage of the disclosed fenestrator devices can be quickly taught, and/or the appearance of one or more components may suggest how the device is to be used. In some embodiments, the disclosed fenestrator devices can be formed using durable material for repeated use. In some embodiments, the disclosed fenestrator devices can be formed using biodegradable materials similar to those used for disposable eating utensils, thereby minimizing the environmental impact of single use products. In some embodiments, the disclosed fenestrator devices can be mass produced at low cost, enabling widespread use by clinics and individual practitioners that cannot afford a costly meshing device.

In some embodiments, the disclosed fenestrator devices can provide the following unique advantages: 1) a support plate can safely and easily separate a graft from perforating elements; 2) the structures of the fenestrator devices can provide positive confirmation that a graft has been perforated by the device; 3) the devices can include a base that supports a graft without contacting perforating elements, thereby preserving their sharpness; 4) the devices can include means for shielding the perforating elements from a user; 5) the devices can include means for separating the components after processing a graft; and 6) the devices can include a plurality of perforating elements having different transverse lengths, thereby reducing user effort or a force required to perforate a graft.

The components, steps, features, objects, benefits and advantages which have been discussed are merely illustrative. None of them, nor the discussions relating to them, are intended to limit the scope of protection in any way. Numerous other embodiments are also contemplated. These include embodiments which have fewer, additional, and/or different components, steps, features, objects, benefits and advantages. These also include embodiments in which the components and/or steps are arranged and/or ordered differently.

The foregoing description details certain embodiments of the systems, devices, and methods disclosed herein. It will be appreciated, however, that no matter how detailed the foregoing appears in text, the devices and methods can be practiced in many ways. As is also stated above, it should be noted that the use of particular terminology when describing certain features or aspects of the invention should not be taken to imply that the terminology is being re-defined herein to be restricted to including any specific characteristics of the features or aspects of the technology with which that terminology is associated. The scope of the disclosure should therefore be construed in accordance with the appended claims and any equivalents thereof.

It will be appreciated by those skilled in the art that various modifications and changes may be made without departing from the scope of the described technology. Such modifications and changes are intended to fall within the scope of the embodiments, as defined by the appended claims. It will also be appreciated by those of skill in the art that parts included in one embodiment are interchangeable with other embodiments; one or more parts from a depicted embodiment can be included with other depicted embodiments in any combination. For example, any of the various components described herein and/or depicted in the Figures may be combined, interchanged or excluded from other embodiments.

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

Unless otherwise stated, all measurements, values, ratings, positions, magnitudes, sizes, and other specifications which are set forth in this specification, including in the claims which follow, are approximate, not exact. They are intended to have a reasonable range which is consistent with the functions to which they relate and with what is customary in the art to which they pertain.

The phrase “means for” when used in a claim is intended to and should be interpreted to embrace the corresponding structures and materials which have been described and their equivalents. Similarly, the phrase “step for” when used in a claim is intended to and should be interpreted to embrace the corresponding acts which have been described and their equivalents. The absence of these phrases in a claim means that the claim is not intended to and should not be interpreted to be limited to any of the corresponding structures, materials, or acts or to their equivalents.

The scope of protection is limited solely by the claims which now follow. That scope is intended and should be interpreted to be as broad as is consistent with the ordinary meaning of the language which is used in the claims when interpreted in light of this specification and the prosecution history which follows and to encompass all structural and functional equivalents.

Except as stated immediately above, nothing which has been stated or illustrated is intended or should be interpreted to cause a dedication of any component, step, feature, object, benefit, advantage, or equivalent to the public, regardless of whether it is or is not recited in the claims.

What is claimed is:

1. A device for fenestrating a graft, the device comprising:
a perforation component having a plurality of perforating elements extending from a body and at least one alignment element extending from said body;
a base component having a plurality of receiving spaces each being configured to at least partially receive at least one of the plurality of perforating elements,
wherein the at least one alignment element is configured to align the perforation component relative to the base component such that the plurality of perforating elements are aligned with the plurality of receiving spaces in at least a transverse direction.

2. The device of claim 1, wherein the plurality of receiving spaces are sized and shaped so as to receive the at least one of the plurality of perforating elements without contacting the at least one of the plurality of perforating elements.

3. The device of claim 1, wherein the plurality of perforating elements comprise a first set of perforating elements and a second set of perforating elements, wherein a maximum transverse dimension of each of the first set of perforating elements is greater than a maximum transverse dimension of each of the second set of perforating elements.

4. The device of claim 1, wherein the at least one alignment element comprises a dowel.

5. The device of claim 4, wherein the base component comprises an alignment receptacle configured to receive at least a portion of the dowel.

6. The device of claim 1, wherein the at least one alignment element comprises a sleeve, wherein the sleeve is sized and shaped so as to slide over at least a portion of the base.

7. The device of claim 1, further comprising a support plate having a plurality of holes each being configured to receive at least one of the perforating elements therethrough, wherein the at least one alignment element is configured to align the perforation component relative to the support plate such that the plurality of perforating elements are aligned with the plurality of holes in at least a transverse direction.

8. The device of claim 7, wherein the support plate is sized and shaped so as to be disposed between the perforation component and the base component at least when the perforating elements are at least partially received within the receiving spaces.

9. The device of claim 8, wherein the perforation component comprises at least one slot, wherein the support plate comprises at least one tab, and wherein the at least one tab is configured to extend through the at least one slot so as to extend beyond the perforation component.

10. The device of claim 8, wherein the perforation component has a maximum lateral dimension that is greater than a maximum lateral dimension of the support plate.

11. The device of claim 1, wherein the plurality of perforating elements each extend in the transverse direction.

12. The device of claim 11, wherein the at least one alignment element extends from the body in the transverse direction.

13. The device of claim 1, wherein the perforation component comprises at least one flange for use in separating the perforation component from the base.

14. A kit comprising:
the fenestrator device of claim 1; and
a graft.

15. The kit of claim 14, wherein the graft comprises a skin graft.

16. The kit of claim 14, wherein the graft comprises a skin substitute material.

17. The kit of claim 14, further comprising instructions for using the fenestrator device.

18. The kit of claim 17, wherein the instructions for using the fenestrator device include indicia formed on the fenestrator device.

19. A method for perforating a graft, the method comprising:
providing a fenestrator device having a perforation component and a base, the perforation component including a plurality of perforating elements extending from a body, the base including a plurality of receptacles each being configured to at least partially receive at least one of the plurality of perforating elements;
providing a graft;
disposing the graft over at least a portion of the base; and
manipulating the perforation component such that at least one of the plurality of perforating elements perforates the graft and extends at least partially through at least one of the plurality of receptacles.

20. The method of claim 19, wherein the graft comprises a skin graft.

21. The method of claim 19, wherein the graft comprises a skin substitute material.

22. The method of claim 19, wherein providing the fenestrator device includes providing a support plate having a plurality of holes each being configured to receive at least one of the perforating elements therethrough, and wherein manipulating the perforation component includes disposing the perforation component relative to the support plate such that the support plate is disposed between the graft and the perforation component.

23. The method of claim 19, further comprising aligning the perforation component relative to the base such that the plurality of perforating elements are each aligned relative to the plurality of receptacles in at least a transverse direction.

24. The method of claim 23, further comprising perforating the graft with at least one of the plurality of perforating elements.
